(19) United States
(12) Patent Application Publication
(10) Pub. No.: US 2023/0122922 A1
(43) Pub. Date: Apr. 20, 2023

(54) Implant With Ceramic Joint Surface

(71) Applicant: FX SOLUTIONS, BOURG-EN-BRESSE (FR)

(72) Inventor: Jean-Marie DAUDET, BOURG-EN-BRESSE (FR)

(21) Appl. No.: 17/965,487

(22) Filed: Oct. 13, 2022

(30) Foreign Application Priority Data
Oct. 15, 2021 (FR) 2110993
Oct. 29, 2021 (FR) 2111538

(51) Int. Cl. A61F 2/30

(52) U.S. Cl. CPC A61F 2/30; A61F 2002/30405; A61F 2310/00179

(57) ABSTRACT

A joint prosthesis implant includes a main body made mainly of ceramic and comprising a joint outer surface made of ceramic, of concave or convex shape, at least one tightening screw configured to tighten the main body on the patient's bone or on an intermediate element attached to said bone, and at least one added ring in the main body configured to distribute homogeneously in the main body the mechanical stresses caused by tightening the tightening screw.

### CROSS-REFERENCE TO RELATED APPLICATIONS

The present application claims priority from French Provisional Patent Application No. 2110993, filed Oct. 15, 2021 and French Patent Application No. 2111538, filed Oct. 29, 2021, each of which is hereby incorporated by reference herein in its entirety.

### TECHNICAL FIELD

The invention relates to a joint prosthesis implant. More specifically, the invention relates to a joint prosthesis implant whose joint outer surface is made of ceramic.

### BACKGROUND

The state of the art already describes several types of joint prosthesis, for example shoulder prostheses, hip prostheses and knee prostheses.

These joint prostheses all comprise two implants, each having a joint outer surface intended to come into contact with each other, thus reproducing the joint. To reproduce the best possible joint quality, it is known to use pairs of favorable materials for these joint outer surfaces in contact, these pairs possibly consisting of two “hard” materials or one “hard” material and one “soft” material. The “hard” materials known and used in joint prosthesis implants are the cobalt-chromium alloy, titanium-stainless steel alloy and ceramic. The “soft” materials may be plastic, for example ultra-high-molecular-weight polyethylene (UHMWPE) or polyetheretherketone (PEEK) or polyetherketoneketone (PEKK) and their derivatives.

The cobalt-chromium alloy is particularly appreciated by joint prosthesis implant manufacturers since it offers exceptional tribological and mechanical properties which allowed in particular the implant concerned to be screwed and tightened (using one or more screws located in the implant) either directly into the patient's bone, or in an element itself attached to the patient's bone (for example a metaglene for a reverse total shoulder prosthesis), with no risk of damaging the implant.

However, the latest applicable standards (and in particular the updated standard of the delegated regulation (UE) 2020/217 of 4 Oct. 2019 REACH) tend to prohibit the use of chromium cobalt as material for the joint prosthesis implants since cobalt is considered to be a potentially carcinogenic, mutagenic or toxic substance for the patient. Alternative hard materials must therefore be considered.

One known hard material is ceramic. This material offers the advantage of being biocompatible and especially suitable for rubbing due to its hardness properties. However, ceramic is a material with very low ductility and the very high, localized mechanical stresses caused by the above-mentioned tightening using one or more screws could create cracks in the ceramic, or even break it. Such a risk is not acceptable, whether for the surgeon during the operation or for the patient who could need to have the prosthesis replaced too quickly. In addition, it is not currently authorized to install another ceramic implant when the ceramic of a first implant has broken. In this case therefore, another pair of materials must be chosen.

### SUMMARY

The invention aims in particular to allow the use of such a ceramic implant which overcomes all the above-mentioned disadvantages.

This invention therefore relates to a joint prosthesis implant comprising:
a main body made mainly of ceramic and comprising a joint outer surface made of ceramic, of concave or convex shape,
at least one tightening screw configured to tighten the main body on the patient's bone or on an intermediate element attached to said bone,
at least one added ring in the main body and forming with it a chamber trapping the head of the tightening screw and comprising a bearing surface forming a stop for said head of the tightening screw, the added ring being configured to be in contact with the tightening screw during said tightening, the added ring comprising a central hole crossed by the tightening screw, the added ring being configured to distribute homogeneously in the main body the mechanical stresses caused by tightening the tightening screw.

Thus, a joint prosthesis implant made of ceramic (or mainly of ceramic) that is both functional and robust and that is not likely to crack or break when tightening the tightening screw can therefore be produced. The presence of the added ring offers the twofold advantage of helping to trap the screw head, thus limiting its movements, and providing a bearing surface against which the head of the tightening screw presses when the tightening screw is being tightened. Due to the contact between the added ring and the tightening screw, the mechanical stresses caused by tightening the tightening screw are distributed homogeneously in the main ceramic body, thereby preventing these forces from being transmitted to specific areas which would lead to the appearance of cracks in the main body or break it.

The added ring can be made of any material allowing the mechanical stresses to be evenly distributed in the main body. The added ring can in particular be made entirely or partially of metal, plastic or ceramic. It can also be made of several different materials, including a composition of the above-mentioned materials.

Homogeneous distribution of the mechanical stresses caused by tightening the screw means that the stresses are distributed over the entire surface of the main body in contact with the added bush, thereby preventing a stress which would be greater than the elastic limit and which would break the ceramic from being applied on the main body. Due to the large radial contact area between the added ring and the main body, the forces are distributed over a larger area of the main body so that the localized stresses are as low as possible.

According to other optional characteristics of the invention taken alone or in combination:
the added ring comprises a threaded portion, the main body comprises a corresponding tapped portion, the threaded portion and the tapped portion being configured so that the added ring can be screwed into the main body;
the added ring comprises means for clipping to the main body;
the added ring comprises elastically deformable peripheral means configured to deform as said tightening screw is inserted;
the added ring is split so that it can expand as said tightening screw is inserted;
the added ring is made of a thermally expansive material exerting expansion pressure on the main body;
the added ring is added by brazing onto the main body;
the added ring is bonded onto the main body;
the main body is a glenosphere, the tightening screw being configured to tighten the glenosphere onto a metaglene attached to the patient's glenoid;
the diameter of the glenosphere is between 30 and 46 mm;
the main body is a ceramic tibial plateau, a knee condyle, an anatomic glenoid, a hip acetabulum or a ceramic cup.

The invention also relates to a joint prosthesis comprising an implant according to any one of the preceding variants of the invention.

### BRIEF DESCRIPTION OF THE DRAWINGS

The invention will be better understood on reading the following description, given solely by way of example and with reference to the accompanying drawings in which:

FIG. 1 is an exploded view (FIG. 1A) and a cross-sectional view (FIGS. 1B and 1C) of a first variant of the first embodiment where the ring is screwed into the housing, located in the dome of the ceramic glenosphere,

FIG. 2 is an exploded view (FIG. 2A) and a cross-sectional view (FIGS. 2B and 2C) of a second variant of the first embodiment where the ring is clipped into the housing, located in the dome of the ceramic glenosphere,

FIG. 3 is an exploded view (FIG. 3A) and a cross-sectional view (FIGS. 3B and 3C) of a third variant of the first embodiment where the ring is also clipped into the housing, located in the dome of the ceramic glenosphere,

FIG. 4 is an exploded view (FIG. 4A) and a cross-sectional view (FIGS. 4B and 4C) of a fourth variant of the first embodiment where the ring is added by expansion fitting into the housing, located in the dome of the ceramic glenosphere,

FIG. 5 is an exploded view (FIG. 5A) and a cross-sectional view (FIGS. 5B and 5C) of a fifth variant of the first embodiment where the ring is added by brazing into the housing, located in the dome of the ceramic glenosphere,

FIG. 6 consists of two cross-sectional views showing a position in which the implant is free (FIG. 6A) and a position in which the implant is tightened (FIG. 6B) according to the second variant of the first embodiment,

FIG. 7 consists of two cross-sectional views of a first variant of the second embodiment comprising two rings (only one is shown), each one being clipped into a housing located in the anatomic glenoid, showing a position in which the implant is free (FIG. 7A) and a position in which the implant is tightened (FIG. 7B).

### DETAILED DESCRIPTION

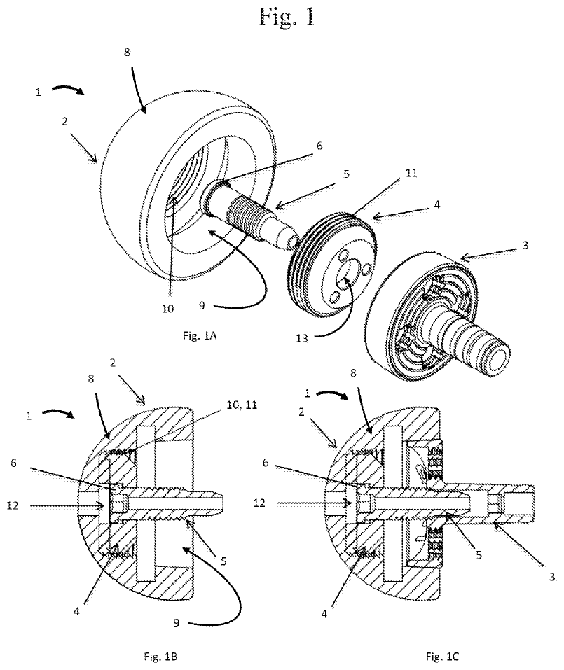

FIGS. 1A to 6B show a joint prosthesis implant according to a first embodiment of the invention, itself described according to several variants, the implant according to this first embodiment being designated by the general reference 1.

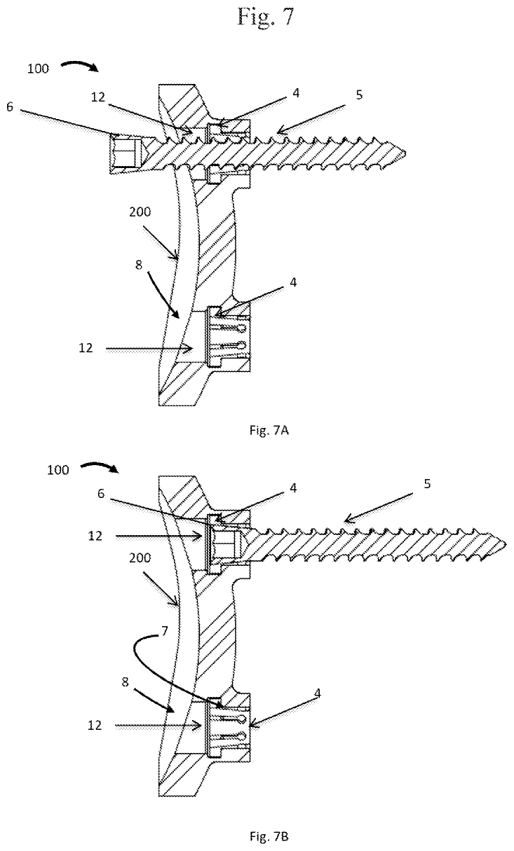

FIGS. 7A and 7B show a joint prosthesis implant according to a second embodiment of the invention, the implant according to this first embodiment being designated by the general reference 100.

For these two embodiments shown, as well as for those not shown, the joint prosthesis implant 1 or 100 comprises:
a main body 2 or 200 made mainly of ceramic and comprising a joint outer surface 8 made of ceramic, of concave or convex shape,
at least one tightening screw 5 configured to tighten the main body 2 or 200 on the patient's bone (not shown) or on an intermediate element 3 attached to said bone,
at least one added ring 4 in the main body 2 or 200 and forming with it a chamber 12 trapping the head 6 of the tightening screw 5 and comprising a bearing surface forming a stop for said screw head, the ring being configured to be in contact with the screw 5 during said tightening, the ring 4 comprising a central hole 13 crossed by the screw 5, the ring 4 being configured to distribute homogeneously in the main body 2 or 200 the mechanical stresses caused by tightening the screw 5.

Note that the variants shown for the first embodiment, in which the implant is a glenoid implant for reverse shoulder prosthesis, can apply to any prosthesis comprising a joint implant having a ceramic joint surface and being screwed and tightened using at least one tightening screw to the patient's bone or to an intermediate element attached to said bone, as is the case in particular with the second embodiment shown, in which the implant is a glenoid implant for anatomic shoulder prosthesis comprising an added ring 4 corresponding to that of the second variant of the first embodiment.

Any type of ceramic known by those skilled in the art can be used for the main body. For example, it could be alumina, zirconia or a composite material. Since ceramic is a hard material, it can be used with pairs of “hard/hard” or “hard/soft” materials. In this second example, the “soft” material can be ultra-high-molecular-weight polyethylene (UHMWPE) or polyetheretherketone (PEEK) or polyetherketoneketone (PEKK) or their derivatives.

In the first embodiment (FIGS. 1A to 6B), the main body 2 corresponds to a glenosphere 2 which has a tightening screw 5 used to tighten it to an intermediate element 3 attached to the bone, in this case the metaglene 3.

The ceramic glenosphere 2 can have a diameter from 30 to 46 mm, it can be centered or off-centered and can be lateralized if necessary.

The glenosphere 2 and the metaglene 3 are assembled together using the tightening screw 5. To make the assembly even better, a Morse cone (not shown) is provided between the glenosphere 2 and the metaglene 3.

The glenosphere 2 comprises a housing 9 dimensioned to receive the added ring 4 and forming with it the chamber 12 trapping the head 6 of the tightening screw 5.

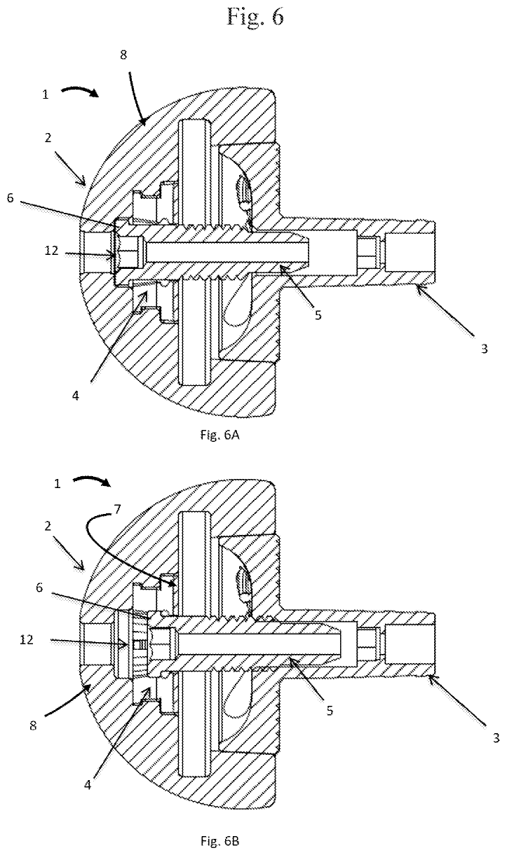

According to a first variant of this embodiment of the invention (FIGS. 1A to 1C), applicable to all the embodiments, the added ring 4 is screwed into the housing 9 of the glenosphere 2 which therefore comprises a tapping 10 of shape complementary to the external radial thread 11 of the added ring 4. When the added ring 4 is in the final position in the housing 9 of the glenosphere 2 (in other words when it has been screwed fully in), it forms with the housing a chamber 12 which traps the head 6 of the tightening screw 5, thus limiting its movements. The added ring 4 has a bearing surface 7 (visible on FIG. 6B) against which the head 6 of the tightening screw 5 presses when the glenosphere 2 is tightened on the metaglene 3. Thus, the forces caused by this tightening are distributed homogeneously over the entire contact surface between the housing 9 of the glenosphere 2 and the added ring 4. This large area which comprises in particular the entire radial area of the added ring minimizes the force of the stresses undergone locally by the ceramic glenosphere 2, which reduces the risk of cracking or breaking the glenosphere.

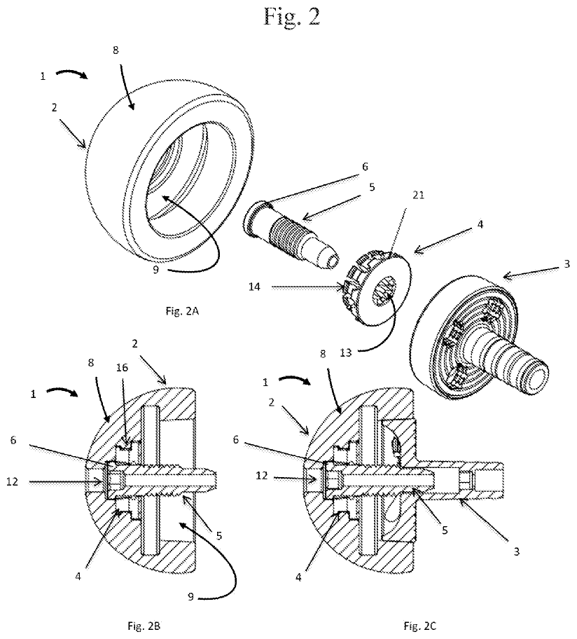

According to a second variant of this embodiment of the invention (FIGS. 2A to 2C), applicable to all the embodiments, the added ring 4 is clipped into the housing 9 of the glenosphere using clipping means thus forming with the housing 9 the chamber 12 trapping the head 6 of the tightening screw 5. More precisely, in this variant, the clipping means consist of teeth 14 having fins 21 at the ends, forming together a groove, and of a tab 16 provided inside the housing 9, shaped to receive the groove formed by the teeth 14 in order to clip the added ring 4. As for all the embodiments and variants of the invention, the added ring 4 comprises a bearing surface 7 forming a stop for the head 6 of the screw 5 when tightening the screw. According to this variant, the added ring 4 comprises elastically deformable peripheral means 21 configured to deform as said tightening screw 5 is inserted (the final position of the tightening screw when it has been screwed fully in is shown on FIG. 6B). These deformable peripheral means 21 may comprise deformable fins 21 which expand on the outside when they are stressed as the tightening screw 5 is inserted, pressing homogeneously on the inner side of the housing 9 of the ceramic glenosphere 2. Thus, as mentioned for the first variant, stresses caused by tightening the glenosphere 2 on the metaglene 3 are distributed more homogeneously, which finally reduces the force of the stresses undergone locally by the ceramic glenosphere 2, reducing the risk of cracking or breaking the glenosphere.

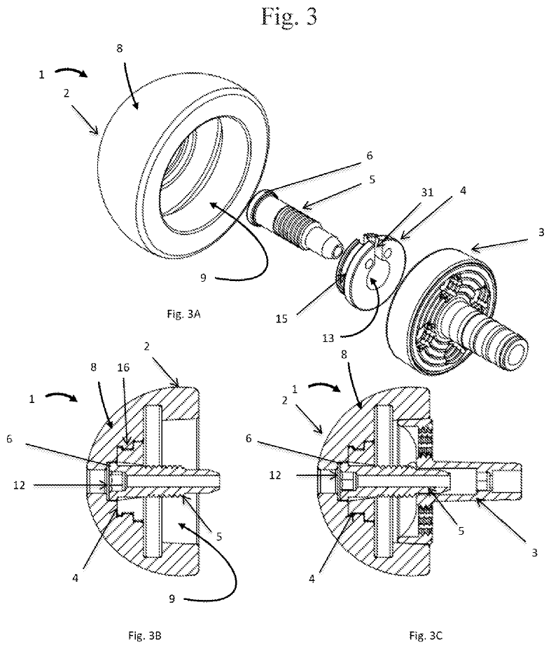

According to a third variant of this embodiment of the invention (FIGS. 3A to 3C), applicable to all the embodiments, the added ring 4 is clipped into the housing 9 of the glenosphere 2 using clipping means thus forming with the housing 9 the chamber 12 trapping the head 6 of the tightening screw 5. More precisely, in this variant, the clipping means consist of a groove 15 (present on the added ring 4) and of a tab 16 (provided inside the housing 9), shaped to receive the groove 15 in order to clip the added ring 4. As for all the embodiments and variants of the invention, the added ring 4 comprises a bearing surface 7 forming a stop for the head 6 of the tightening screw 5 when tightening the screw. According to this variant, the added ring 4 is split 31. This split ring 4 is therefore U-shaped, allowing it to expand on the outside as the tightening screw 5 (having a portion of diameter greater than or equal to the diameter of the central hole 13 of the added ring) is inserted. Thus, the added ring can press homogeneously on the inner side of the housing 9 of the ceramic glenosphere 2, when tightening the tightening screw 5. Once again, the stresses caused by tightening the glenosphere 2 on the metaglene 3 can be distributed more homogeneously.

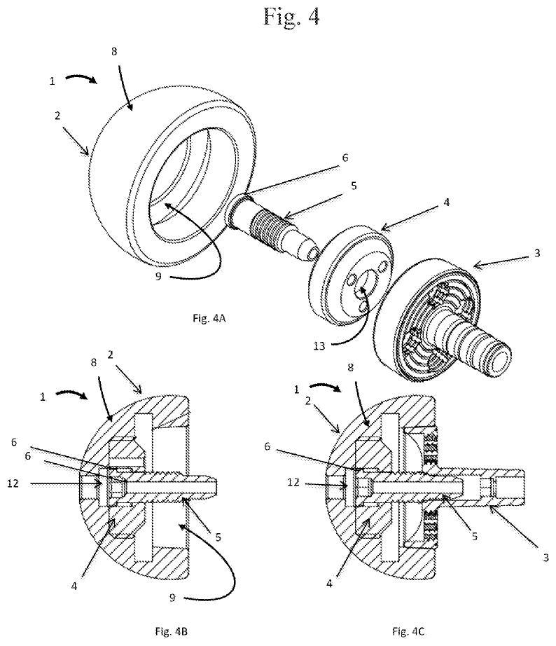

According to a fourth variant of this embodiment according to the invention (FIGS. 4A to 4C), applicable to all the embodiments, the added ring 4 is made of a thermally expansive material exerting expansion pressure inside the housing 9 of the glenosphere 2 after being positioned in the housing. To do this, the added ring 4 can be cooled (in nitrogen for example) and the ceramic glenosphere can be heated so that the added ring 4 can be inserted and positioned correctly in the housing 9 of the glenosphere. The diameter of the ring 4 can be slightly greater than that of the housing of the glenosphere 2 when they are at the same temperature, which means that when the added ring 4 warms up, it exerts a homogeneous compression force over the entire surface 41 of the housing 9 of the glenosphere 2 with which it is in contact. Like all the variants previously described, the stresses caused by tightening the glenosphere 2 on the metaglene 3 can be distributed more homogeneously.

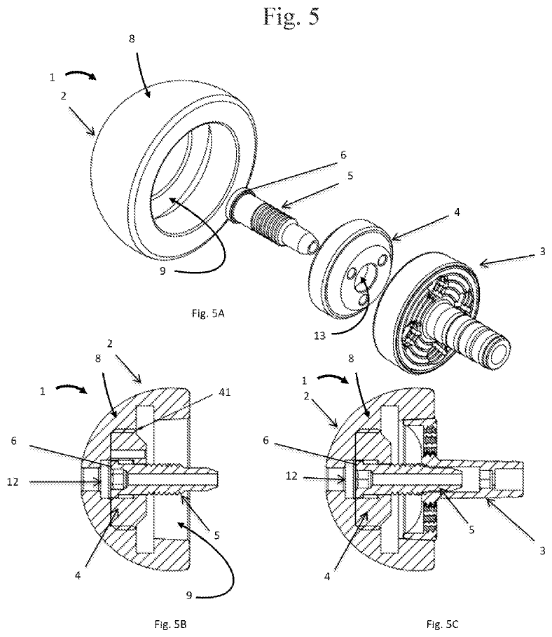

According to a fifth variant of this embodiment of the invention (FIGS. 5A to 5C), the ring 4 is added by brazing into the housing 9 of the glenosphere 2 (a permanent connection 41 is created between the outer surface of the ring 4 and the housing 9 by placing a metal wire between these two elements and heating the wire sufficiently to create said connection). As for the preceding variants, once the ring 4 has been positioned in the housing 9 of the glenosphere 2, it forms with the housing a chamber 12 trapping the head 6 of the tightening screw 5. Like the preceding variants, the added ring 4 according to this variant also comprises a bearing surface 7 forming a stop for the head of the tightening screw.

According to a sixth variant of this embodiment of the invention (not shown), the added ring 4 is bonded in the housing 9 of the glenosphere 2 and thus offers the same advantages as regards the distribution of the stresses caused by tightening as those mentioned in the preceding paragraphs.

In the second embodiment (FIGS. 7A to 7B), the main body 200 corresponds to an anatomic glenoid 200 of an “anatomic” shoulder prosthesis. In this embodiment, the implant 100 comprises at least two tightening screws 5, to tighten directly with the patient's bone, and at least two added rings 4 (similar to that described in the variant shown on FIGS. 2A to 2C). The operation of the two added rings 4 is the same as that described for the first embodiment (where the main body 2 is a glenosphere 2). All the variants described in the first embodiment apply to this second embodiment, as well as to all possible embodiments where the joint implant has a ceramic joint surface and is screwed and tightened using at least one tightening screw, on the patient's bone or on an intermediate element attached to said bone. Each added ring 4 forms with a dedicated housing 9 of the anatomic glenoid 200 a chamber 12 trapping the head 6 of one of the two tightening screws 5. In this case, each tightening screw 5 tightens the anatomic glenoid 200 to the patient's bone (not shown). Thus, as mentioned for all the variants of the first embodiment, stresses caused by tightening the glenoid 200 on the patient's bone are distributed more homogeneously, which finally reduces the force of the stresses undergone locally by the ceramic glenoid 200, reducing the risk of cracking or breaking the glenoid.

Element 41 can refer to a brazing connection. Element 100 can refer to an implant according to the second embodiment.

The invention is not limited to the embodiments described. As mentioned previously, the invention can be applied to any type of joint implant having a ceramic joint surface and being screwed and tightened using at least one tightening screw, on the patient's bone or on an intermediate element attached to said bone. For example, the invention also relates to implants for hip or knee prostheses.

1. A joint prosthesis implant comprising:
a main body made mainly of ceramic and comprising a joint outer surface made of ceramic, of concave or convex shape,
at least one tightening screw configured to tighten the main body on the patient's bone or on an intermediate element attached to said bone,
at least one added ring in the main body and forming with it a chamber trapping the head of the tightening screw and comprising a bearing surface forming a stop for said head of the tightening screw, the added ring being configured to be in contact with the tightening screw during said tightening, the added ring comprising a central hole crossed by the tightening screw, the added ring being configured to distribute homogeneously in the main body the mechanical stresses caused by tightening the tightening screw.

2. The implant according to claim 1, wherein the added ring comprises a threaded portion and the main body comprises a corresponding tapped portion, the threaded portion and the tapped portion being configured so that the added ring can be screwed into the main body.

3. The implant according to claim 1, wherein the added ring comprises means for clipping to the main body.

4. The implant according to claim 1, wherein the added ring comprises elastically deformable peripheral means configured to deform as said tightening screw is inserted.

5. The implant according to claim 1, wherein the added ring is made of a thermally expansive material exerting expansion pressure on the main body.

6. The implant according to claim 1, wherein the added ring is bonded onto the main body.

7. The implant according to claim 1, wherein the main body is a glenosphere, the tightening screw being configured to tighten the glenosphere onto a metaglene attached to the patient's glenoid.

8. The implant according to claim 7, wherein the diameter of the glenosphere is between 30 and 46 mm.

9. The implant according to claim 1, wherein the main body is a ceramic tibial plateau, a knee condyle, an anatomic glenoid, a hip acetabulum or a ceramic cup.

10. A joint prosthesis comprising an implant comprising:
a main body made mainly of ceramic and comprising a joint outer surface made of ceramic, of concave or convex shape,
at least one tightening screw configured to tighten the main body on the patient's bone or on an intermediate element attached to said bone,
at least one added ring in the main body and forming with it a chamber trapping the head of the tightening screw and comprising a bearing surface forming a stop for said head of the tightening screw, the added ring being configured to be in contact with the tightening screw during said tightening, the added ring comprising a central hole crossed by the tightening screw, the added ring being configured to distribute homogeneously in the main body the mechanical stresses caused by tightening the tightening screw.

11. The implant according to claim 1, wherein the added ring is split so that it can expand as said tightening screw is inserted.

12. The implant according to claim 1, wherein the added ring is added by brazing onto the main body.
